(19) United States
(12) Patent Application Publication
(10) Pub. No.: US 2013/0224770 A1
(43) Pub. Date: Aug. 29, 2013

(54) Antibody Diluent Buffer

(71) Applicants: Erik Leeming Kvam, Niskayuna, NY (US); Michael John Gerdes, Albany, NY (US)

(72) Inventors: Erik Leeming Kvam, Niskayuna, NY (US); Michael John Gerdes, Albany, NY (US)

(73) Assignee: GENERAL ELECTRIC COMPANY, Schenectady, NY (US)

(21) Appl. No.: 13/408,757

(22) Filed: Feb. 29, 2012

(51) Int. Cl. G01N 33/53; G01N 21/76; G01N 21/64; B82Y 15/00

(52) U.S. Cl. 435/7.92; 435/7.9; 436/501; 435/7.1; 977/773; 977/902

(57) ABSTRACT

The compositions described include an antibody diluent buffer that minimizes the amount of antibody required for immunoassays and further improves the performance of antibody-based assays. The claimed antibody diluent buffer comprises a high-molecular weight neutral polymer and an antibody that specifically binds to an antigen of interest, wherein the antibody remains in solution in the presence of the polymer. Methods for the use of the antibody diluent buffer to detect an antigen, even trace amounts of an antigen, in an antibody-based assay are also disclosed herein.

### FIELD OF THE INVENTION

The present invention relates to compositions and methods for improving the performance of antibody-based assays.

### BACKGROUND

Antibody-based assays (e.g., immunoassays) are widely used biological techniques to detect the presence of an antigen of interest (e.g., generally a macromolecule such as a protein, a protein fragment, a polysaccharide, a DNA molecule, an RNA molecule, etc.) in a sample, such as in a biological sample removed from a patient or animal (e.g., blood, lymph, urine, saliva, sputum, other bodily secretions, cells, and tissue specimens). These immunoassays are used to detect biological antigens such as proteins, protein fragments, chemical, metabolites, polysaccharides, and nucleic acid molecules (e.g., DNA or RNA) that are associated with bacterial, viral, or fungal infections as well as “biomarkers” indicative of various cancers, cardiovascular conditions, or inflammatory diseases. Given the value of antibody-based assays (e.g., immunoassays) in basic scientific research and in medical diagnostic techniques, compositions and methods for improving the performance and lowering the cost of these assays would be beneficial.

Although antibodies and antibody-based assays are powerful tools in the biological arts, a significant obstacle in the performance of immunoassays remains the requirement for large amounts of antibody to perform these methods that leads to high costs associated with the production and purification of antibodies. These costs are magnified in those instances requiring chemical conjugation of a detectable reporting agent such as a fluorescent moiety, an enzyme, a luminescent material, a bioluminescent material, a radioactive material, and nanoparticles. Furthermore, certain antibody-based assays require the use of two antibodies, the first of which is unlabeled and binds to the antigen of interest, and a second antibody that binds to the first antibody, wherein the second antibody comprises a detectable substance that permits the detection of the antigen's presence in the sample.

One way to reduce antibody consumption and lower the cost of antibody-based assays (e.g., immunoassays), is to utilize a lower concentration of antibody; however, this practice often reduces the sensitivity of the antibody-based assay by limiting detection of the antigen as a result of the reduced amount of antibody present to bind to the antigen of interest. Antibody diluent buffers comprising a polyethylene glycol (PEG), with a molecular weight of less than or equal to 20 kD, have been used to improve the performance of ELISAs. Compositions and methods, however, that allow for the use of a lower concentration of antibody in immunoassays while maintaining, or optimally increasing, the sensitivity and overall performance of the antibody-based assay are needed in the art. Furthermore, ideally these compositions and methods would be capable of detecting even trace quantities of an antigen of interest.

### BRIEF DESCRIPTION

The compositions described herein include an antibody diluent buffer that minimizes the amount of antibody required for immunoassays and further improves the performance of these antibody-based assays. The claimed antibody diluent buffer comprises a high-molecular weight neutral polymer that functions as a macromolecular crowding agent and an antibody that specifically binds to an antigen of interest, wherein the antibody is soluble and remains in solution in the presence of the polymer. In particular an aspect of the invention, the high-molecular weight neutral polymer has a molecular weight of less than 25 kD (e.g., PEG-20,000). In other embodiments, the high-molecular weight neutral polymer is a dextran, Ficoll, polyvinyl alcohol (PVA), guar gum, or hydroxyethyl starch (HES). Any high-molecular weight neutral polymers, with the possible exclusion of PEGs, are encompassed by this disclosure. Methods for the use of the antibody diluent buffer to detect an antigen, even trace amounts of an antigen, in an antibody-based assay are also disclosed herein.

### DRAWINGS

These and other features, aspects, and advantages of the present invention will become better understood when the following detailed description is read with reference to the accompanying drawings in which like characters represent like parts throughout the drawings, wherein:

FIG. 1 provides a comparative analysis of PEGs of different molecular weights and concentrations on Cy3-labeled anti-c-Myc signal intensity by slide array immunostaining. Bars represent fold-increase in Cy3 signal relative to the mean of 5 “no PEG” control wells incubated with antibody staining solution alone. Further details are set forth in Examples 1 and 2.

FIG. 2 provides a comparative analysis of antibody signal intensity using antibody diluent buffers comprising PVA (FIG. 2A), guar (FIG. 2B), PVP (FIG. 2C), Ficoll (FIG. 2D), or dextran (FIG. 2E) relative to 6.25% PEG 20,000. Bars represent fold-increase in Cy3 signal relative to the mean of 5 control wells incubated with antibody staining solution alone. Slide array data were averaged from 9 c-Myc peptide spots per dilution (n=3 wells). Further details are set forth in Examples 1 and 3.

FIG. 3 provides a comparative slide array analysis of Cy3-labeled anti-c-Myc signal intensity as a function of c-Myc peptide concentration and using antibody diluent buffers comprising different concentrations of PEG-20,000 (FIG. 3A) or dextran-100 (FIG. 3B). Control data were averaged from 15 peptide spots per dilution (n=5 wells). Experimental details are set forth in Examples 1 and 4.

FIG. 4 provides representative slide array images following immunostaining in the absence or presence of dextran-100 or PEG 20,000 at different antigen concentrations. See Examples 1 and 4.

FIG. 5 provides representative Cy3-labeled anti-c-Myc non-specific background fluorescence following immunostaining in the presence or absence of dextran-100 or PEG 20,000. The area surrounding each immunostained array was sealed by a rubber gasket inside a 96-well Arrayit microplate hardware system. See Examples 1 and 4.

FIG. 6 provides formalin fixed paraffin embedded (FFPE) tissue samples of normal prostate following standard clearing, hydration, and antigen retrieval procedures, followed by immunostaining in antibody diluent buffers with or without dextran-100. Image A was stained with polyclonal pan-cadherin antibody and Cy3-labeled secondary antibody in conventional antibody diluent buffer. Image B was stained with Cy3-conjugated polyclonal pan-cadherin antibody in conventional antibody diluent buffer. Image C was stained with the same antibody as image B but in the presence of antibody diluent buffer comprising 8% (w/v) dextran-100. Experimental details are set forth in Examples 1 and 5.

### DETAILED DESCRIPTION

An antibody diluent buffer comprising a high-molecular weight neutral polymer is provided herein. The skilled artisan would immediately appreciate that the term “high-molecular weight neutral polymer” generally refers to a large molecule (e.g., a macromolecule) comprising repeating structural units and possesses no net charge. The term “polymer” is used herein in its broadest sense to include substances as divergent as plastics, polyesters, polysaccharides, polypeptides (e.g., proteins), and polynucleotides. A “high-molecular weight polymer” is typically one with a molecular weight of 10 kD or greater. A high-molecular weight molecular as used herein may at times refer to one having a molecular weight of less than 25 kD (e.g., PEG-20,000). The high-molecular neutral polymers for use in the instant application include but are not limited to a dextran, Ficoll, polyvinyl alcohol (PVA), guar gum, and hydroxyethyl starch (HES). All high-molecular weight polymers, possibly excluding PEGs are captured by the compositions and methods of the invention. In certain aspects of the invention, the high-molecular weight neutral polymer is a dextran of a particular molecular weight. Although any high-molecular weight dextran may be used in the practice of the invention, the dextran present in the antibody diluent buffer is usually in the range of 10 kD to 150 kD, particularly 40 kD to 100 kD, more particularly 100 kD. Again, without intending to limit the claimed subject matter to a specific high-molecular weight neutral polymer of a certain concentration of such high-molecular weight neutral polymer (e.g. a dextran), a concentration of between 5% (w/v) and 15% (w/v), particularly 6% (w/v) and 13% (w/v), more particularly 6.25% (w/v) to 12.5% (w/v) is utilized in certain aspects of the present application. The high-molecular weight neutral polymers, particularly dextran, encompassed by the invention may be diluted in a buffer including but not limited to phosphate buffered saline (PBS) in order to achieve the desired concentration of the high-molecular weight neutral polymer.

The antibody diluent buffer of the instant application further comprises an antibody, as described in detail below, which specifically binds to an antigen of interest. The antibody present in a claimed antibody diluent buffer is soluble and remains in solution in the presence of the high-molecular weight neutral polymer. As shown in the figures and described in the Examples below, the same antibody concentration led to a two to twenty-fold increase in signal enhancement, which is representative of the amount of antigen bound (e.g., detected) by the antibody, when the claimed antibody diluent buffer comprising a high-molecular weight neutral polymer was used in an antibody-based assay relative to that observed with a “conventional” antibody buffer lacking a high-molecular-weight neutral polymer. Stock concentrations of an antibody of interest may be diluted to produce a specific antibody ratio of interest for use in the claimed methods. One of skill in the art would be able to select an appropriate buffer to prepare antibody dilutions.

In certain aspects of the present disclosure, methods for detecting an antigen in a sample are described, wherein the methods comprise the steps of providing an antibody diluent buffer comprising a high-molecular weight neutral polymer and a primary or secondary antibody, wherein the antibody remain in solution; incubating the antibody diluent buffer with a sample that may comprise the antigen; and determining if the antigen is present in the sample. The high-molecular weight neutral polymer suitable for use in the methods disclosed herein includes but is not limited to a dextran, Ficoll, polyvinyl alcohol (PVA), guar gum, or hydroxyethyl starch (HES). The phrase “high-molecular weight polymer” expressly excludes PEGs in certain compositions and methods disclosed here. The high-molecular weight neutral polymer is optionally a dextran, particularly a dextran with a molecular weight of between 40 kD and 100 kD, more particularly a dextran with a molecular weight of approximately 100 kD.

The term “sample” is used in the instant application to refer to both biological samples that have been provided by a human patient or an animal (e.g., blood, lymph, urine, saliva, sputum, other bodily secretions, cells, and tissue specimens) and non-biological samples (e.g., samples prepared in vitro comprising varying concentrations of an antigen of interest in solution used to assess the usefulness of the compositions and methods disclosed herein).

As used herein, the phrase “determining if the antigen is present in the sample” generally refers to one of numerous methods known by the skilled artisan for detecting binding of an antibody to an antigen. Such techniques include the detection of a labeled substance conjugated to an antibody of this application and are well known in the fields of immunology, molecular biology, and biochemistry.

The claimed methods are intended to improve the performance of an immunoassay. The phrase “improves the performance of an immunoassay” is intended to include a variety of advantageous properties resulting from the use of the antibody diluent buffer of the invention, including but not limited to: minimizing the amount of antibody needed to perform the antibody-based assay, improving the antibody “signal,” permitting the detection a smaller amount (e.g. a “trace” amount) of the antigen in the sample, and maintaining or ideally reducing a reasonable level of non-specific, background binding.

The term “non-specific binding” or “background binding” is a well-known term in the biological arts and generally refers to unintended, passive binding of, for example, an antibody to a substrate used in the assay or to a contaminant present in the assay. In contrast, “specific binding” refers to the desired interaction of an antibody to the appropriate antigen.

Without intending to limit the mechanism of action of the claimed antibody diluent buffers described here, the claimed antibody diluent buffers likely produce the observed results as a consequence of a well-known biophysical and biological concept known as “macromolecular crowding” or “crowding.” This common process in biology occurs when high concentrations of macromolecules in a small volume reduce the solvent accessibility of other large molecules, such as proteins (e.g. antibodies), in solution, thereby artificially increasing the effective concentration of the latter (e.g., a protein, more particularly an antibody). For proteins such as enzymes, crowding is known to alter the kinetics of substrate binding and dissociation and improve enzymatic activity. Therefore, the high-molecular weight neutral polymers (e.g., that result in “macromolecular crowding”) of the antibody diluent buffer disclosed herein effectively increases the concentration of antibody present in the solution relative to “traditional” antibody buffers that contain the same amount of antibody but lack the high-molecular weight neutral polymers.

To more clearly and concisely describe and point out the subject matter of the claimed invention, the following definitions are provided for specific terms, which are used in the following description and in the appended claims. Throughout the specification, exemplification of specific terms should be considered as non-limiting examples.

Antibodies are proteins, more specifically glycoproteins, and exhibit binding specificity to an antigen (e.g., a portion of a macromolecule such as a polypeptide) of interest. The term “antibody” is used in the broadest sense and covers fully assembled antibodies, antibody fragments that can bind antigen (e.g., Fab′, F′(ab)₂, Fv, single chain antibodies, diabodies), and recombinant peptides comprising the foregoing. “Antibody fragments” comprise a portion of an intact antibody, preferably the antigen-binding or variable region of the intact antibody. Examples of antibody fragments include Fab, Fab′, F(ab′)₂, and Fv fragments, diabodies, and linear antibodies (Zapata et al. (1995) Protein Eng. 8(10):1057 1062), single-chain antibody molecules, and multi-specific antibodies formed from antibody fragments. Any monoclonal or polyclonal antibody or antibody fragment capable of binding to an antigen of interest may be used in the practice of the invention.

“Immunoassay” or “antibody-based assay” is used herein in its broadest sense to include any technique based on the interaction between an antibody and its corresponding antigen. The terms immunoassay and antibody-based assay may be used interchangeably in the present application. Such assays are based on the unique ability of an antibody to bind with high specificity to one or a very limited group of similar molecules. The term “antigen” refers to a molecule that binds to an antibody. Immunoassays can be carried out using either the antigen or antibody as the “capture” molecule to “entrap” the other member of the antibody-antigen pairing. As used herein, the term “immunoassay” or “antibody-based assay” further includes those assays that utilize antibodies for the detection of a non-protein biomarker in a biological sample (e.g., nucleic acids or metabolites of biochemical reactions).

An exemplary, albeit not exhaustive list of immunoassays includes a radioimmunoassay (RIA), an enzyme immunoassay (EIA), an enzyme-linked immunosorbent assay (ELISA), a fluorescent immunoassay, and a chemiluminescent immunoassay. One of skill in the art is capable of selecting and implementing the appropriate immunoassay under a particular set of circumstances, as well performing these immunoassays and interpreting their results. Immunoassays may produce qualitative or quantitative results depending on the particular method of detection selected.

A variety of immunoassays exist in the art, including those for drug testing, hormones, numerous disease-related proteins, tumor protein biomarkers, and protein biomarkers for cardiac injury. Immunoassays are also used to detect antigens on infectious agents such as Hemophilus, Cryptococcus, Streptococcus, Hepatitis B virus, HIV, Lyme disease, and Chlamydia trichomatis. These immunoassay tests are commonly used to identify patients with these and other diseases. Accordingly, compositions and methods for improving the sensitivity, specificity, and detection limits in immunoassays are of great importance in the field of diagnostic medicine.

In certain aspects of this invention, determining if an antigen is present in a sample requires detection of antibody binding to the antigen. Any method known in the art for detecting antibody binding is encompassed by the disclosed invention. The determination and optimization of appropriate antibody binding detection techniques is standard and well within the routine capabilities of one of skill in the art. In some embodiments, detection of antibody binding to an antigen of interest can be facilitated by coupling the antibody to a detectable substance. Examples of detectable substances include various enzymes, prosthetic groups, fluorescent materials, luminescent materials, bioluminescent materials, radioactive materials, and nanoparticles. Exemplary suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin; a detectable luminescent material that may be couple to an antibody includes but is not limited to luminol; examples of bioluminescent materials include luciferase, luciferin, and aequorin; examples of suitable radioactive material for detection of antibody binding include ¹²⁵I, ¹³¹I, ³⁵S, or ³H; examples of nanoparticles include colloidal gold or carbon nanotubes. Methods for conjugating or coupling a detectable substance to an antibody and for detecting these agents are well known in the art.

### EXAMPLES

### Example 1

### Description of Experimental Protocols Used to Analyze Antibody Diluent Buffers

### Peptide Microarray Design and Preparation

Blocking peptides used in this study were c-Myc peptide (M2435, Sigma, St. Louis, Mo.) and Phospho-CREB (pCREB-Ser133) peptide (#1090, Cell Signaling Technology, Danvers, Mass.). pCREB was used as a negative control in this example.

A Spotbot 2 microarrayer (Arrayit Corp., Sunnyvale, Calif.) was used to print each peptide microarray in a pattern conducive for a 4×24 microplate hardware system (Arrayit Corp.). Blocking peptides were diluted in pH 8.5 buffer at different concentrations and spotted at room temperature (45-50% relative humidity) using a SMP3 pin. The peptides were covalently bound to NHS ester groups on Codelink HD modified glass slides (Surmodics IVD, Eden Prairie, Mn.). Printed slides were stored at 4° C. prior to use. Peptides were printed in triplicate spots in each microarray. A schematic of the printed 3×10 array is shown in Table 1. A total of 24 wells were printed on a slide, each containing a 3×10 array.

TABLE 1
Peptide Spots Printed in 3 × 10 Arrays

| Row | Target | Spotting concentration | |
|---|---|---|---|
| 1 | pCREB peptide | 0.1 | mg/mL |
| 2 | Pin wash (pCREB) | Trace | |
| 3 | c-Myc peptide | 0.00625 | mg/mL |
| 4 | c-Myc peptide | 0.0125 | mg/mL |
| 5 | c-Myc peptide | 0.025 | mg/mL |
| 6 | c-Myc peptide | 0.05 | mg/mL |
| 7 | c-Myc peptide | 0.1 | mg/mL |
| 8 | c-Myc peptide | 0.2 | mg/mL |

| | | |
|---|---|---|
| 9 | Pin wash (c-Myc) | Trace |
| 10 | Pin wash (c-Myc) | Trace |

Initial quality control experiments demonstrated significant variability in the peptide printing process, including slide-to-slide “drift” in multiplex formats as well as stochastic intra-well variability in individual peptide dilution spots. To mitigate the risks posed by such variability, arrays were printed on a single-slide basis using a single pin loading technique.

### Polymer Crowding Agents

Synthetic and natural polymers were purchased from Sigma-Aldrich and GE Healthcare as suspensions in water, when available. Dry polymers were suspended in sterile water and mixed under gentle heat until a clarified solution was obtained. The following table lists the stock concentrations of crowding agents tested in this study:

TABLE 2
Stock concentrations of Crowding Agents

| Polymer | Concentration (w/v) |
|---|---|
| Dextran-40 (40,000 Da) | ~25% |
| Dextran-100 (100,000 Da) | ~25% |
| Ficoll PM70 (Mᵣ ~70,000) | ~50% |
| Ficoll PM400 (Mᵣ ~400,000) | ~50% |
| Guar | ~3% |
| PEG 4,000 | ~50% |
| PEG 8,000 | ~50% |
| PEG 20,000 | ~50% |
| PVP (M_{w} 360,000) | ~15% |
| PVA (M_{w} 146,000-186,000) | ~5% |

With the exception of dextran, the stock concentrations listed in the table above reflect the maximum workable concentration in which the solution could be pipetted. In contrast to most of the polymers listed, guar exhibited the least solubility in water and remained as a heterogeneous suspension even after heating.

### Immunostaining

Slides containing printed c-Myc peptide arrays were sandwiched into an Arrayit 96-well microplate hardware system, which accommodates up to four glass slides under a 96-well formatted gasket and housing. This system was assembled to ensure that each microplate well contained a single printed array. For immunostaining, 100 μL of blocking solution (10% donkey serum, 3% BSA in PBS) was pipetted into each well and incubated for approximately two hours at 4° C. Blocking solution was carefully aspirated from each well and replaced with 100 μL antibody staining solution (monoclonal anti-c-Myc-Cy3 diluted 1:300 in PBS+3% BSA). Polymeric crowding reagents were serially diluted in PBS from the stock concentrations listed in Table 3 and mixed with an equal volume of concentrated antibody staining solution (anti-c-Myc-Cy3 diluted 1:150 in PBS+3% BSA) to yield a final antibody solution of 1:300. Antibody was incubated on the array for 1 hour at room temperature (wrapped in parafilm and shielded from direct light). Wells were carefully aspirated and washed 3×5 min with 200 μL. PBS. To mitigate the risks posed by microarray printing variability, immunostaining experiments were conducted in triplicate wells oriented diagonally to reduce positional bias.

### Imaging

Following immunostaining, slides were removed from the Arrayit 96-well housing and carefully aspirated of any residual PBS pooled on the glass surface. The slides were placed into a multi-slide tray and scanned on a Typhoon Variable Mode Imager to capture Cy3 fluorescence on the array. Images were analyzed with ImageQuant TL using the array analysis tool. Peptide spots were defined using a microarray grid, and the fluorescence intensity of each spot was quantified after background subtraction.

### Example 2

### Analysis of PEG Comprising Antibody Diluent Buffers

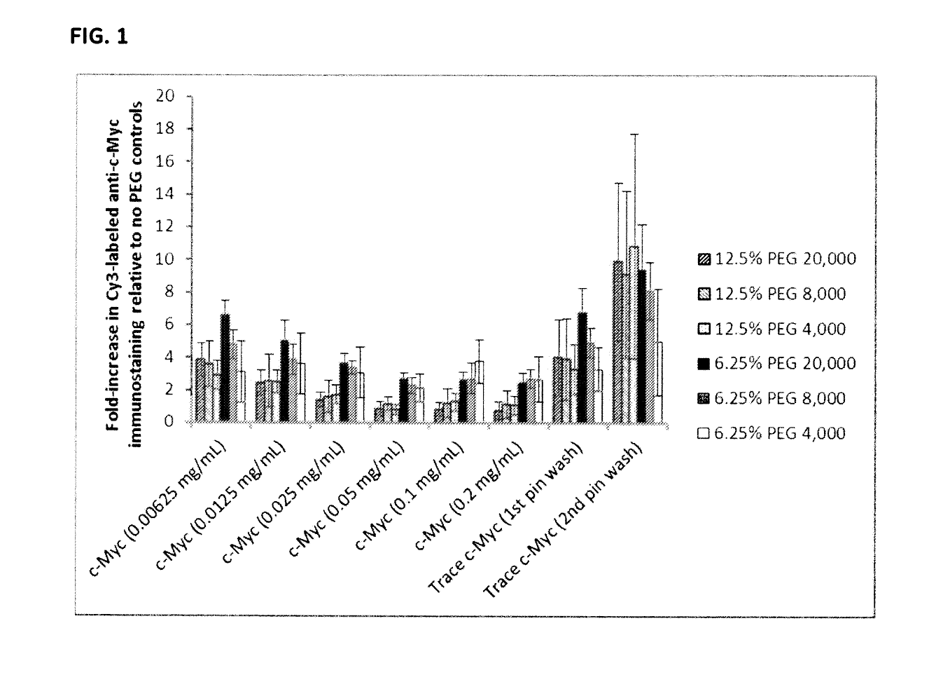

PEG solutions of three different molecular weights of 20 kD or less (e.g., 4 kD, 8 kD, and 20 kD) were screened for immunostaining of Cy3-labeled anti-c-Myc monoclonal antibody on c-Myc blocking peptide microarrays, essentially as described above. Briefly, antibody staining solutions containing fixed concentrations of PEG (6.25% and 12.5% (w/v)) were tested in triplicate wells oriented diagonally on the slide array to reduce positional bias and mitigate any variability introduced during the peptide printing process. In doing so, Cy3 fluorescence was averaged over three different microarrays distributed “randomly” on the slide. To standardize the results in order to draw comparisons to future experiments, the magnitude of Cy3 fluorescence was normalized to antibody control wells lacking PEG and used to calculate a “relative fold-increase.” As shown in FIG. 1, PEG-20,000 provided the highest signal output at 6.25% (w/v) relative to 12.5% (w/v) or analogous concentrations of smaller molecular weight PEGs. Lower molecular weight PEGs displayed smaller fold-increases. Relative fold-increases in antibody staining varied from approximately 2 to 12-fold depending on the c-Myc antigen concentration. The greatest fold-increase was observed using c-Myc peptide concentrations below 0.025 mg/mL in the presence of 6.25% PEG 20,000.

For all concentrations of PEG tested, no immunostaining was observed at pCREB peptide spots (e.g., negative control), thus indicating that the substrate-specificity of the anti-c-Myc antibody was maintained in the presence of the PEG reagent. Non-specific, background Cy3 fluorescence, however, generally increased on the slide surface as a function of PEG molecular weight and concentration, consistent with macromolecular crowding theory.

### Example 3

### Comparison of Antibody Diluent Buffers Comprising Ficoll, Guar Gum, Polyvinyl Alcohol (PVA), Polyvinylpyrrolidone (PVP), or Dextran, to PEG

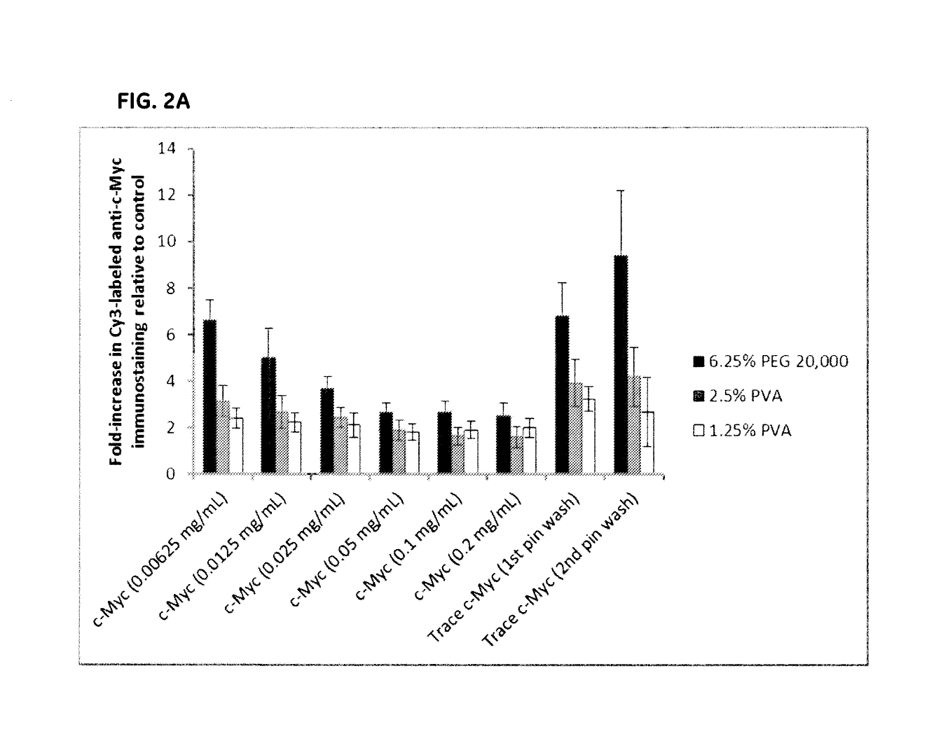

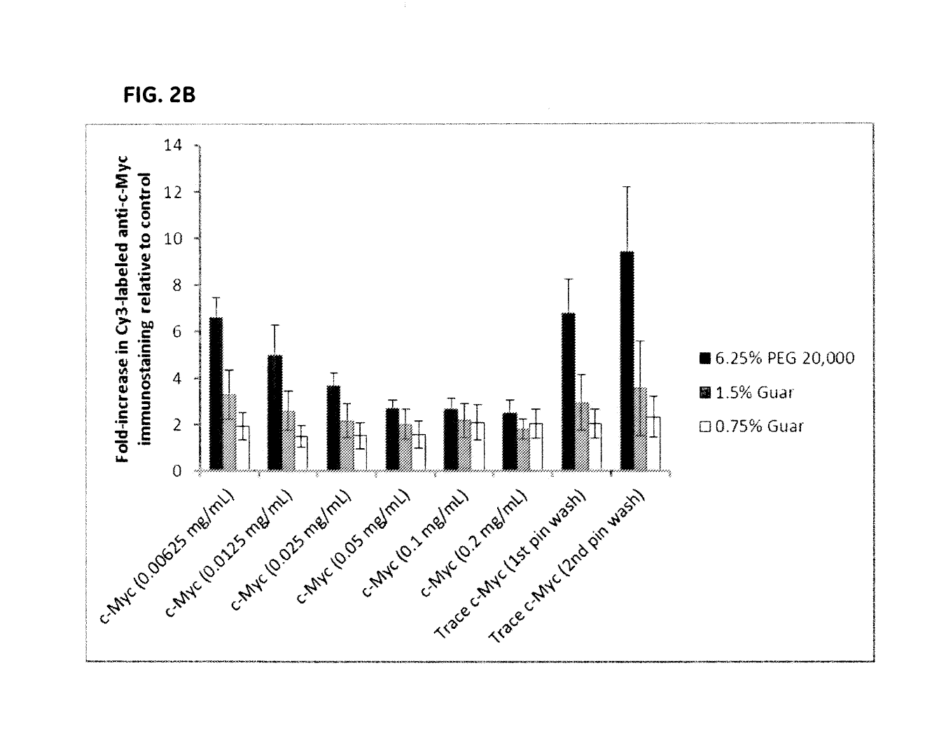

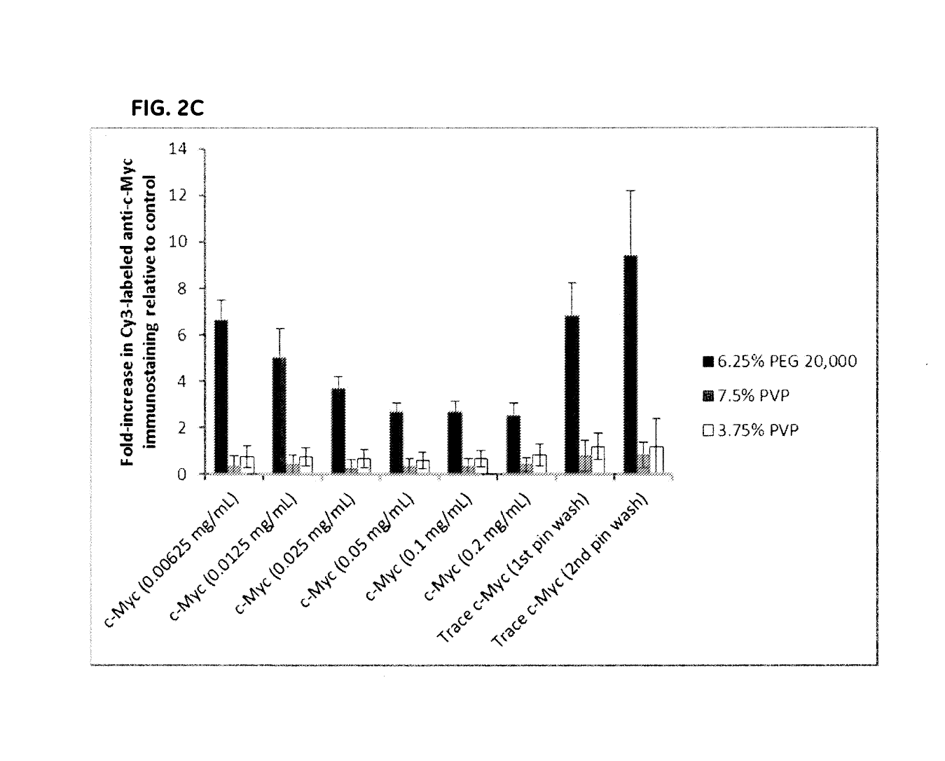

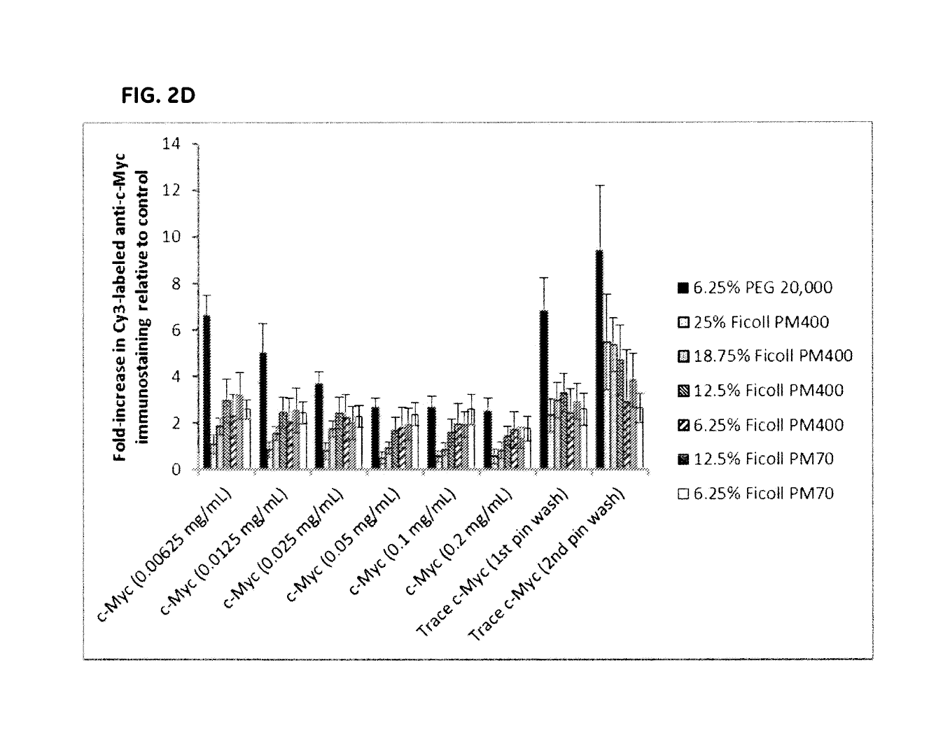

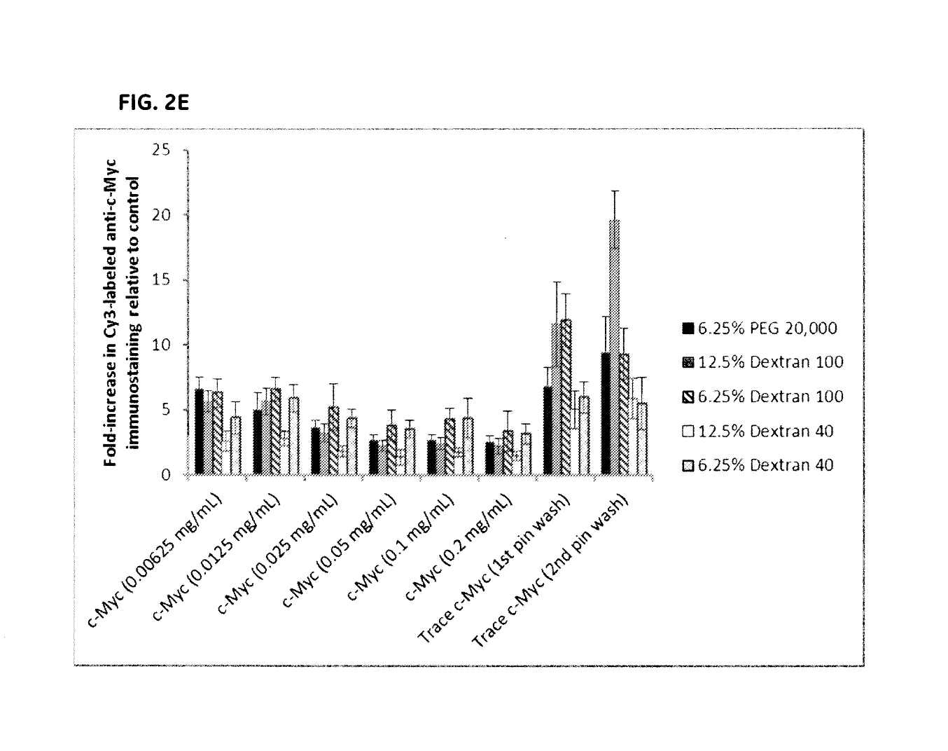

Using the slide array procedure outlined above, Cy3-labeled anti-c-Myc immunostaining was evaluated and compared among different concentrations of Ficoll, guar gum, PVA, PVP, or dextran on printed c-Myc peptide arrays. Cy3 fluorescence was normalized to antibody control wells lacking any of the above high-molecular weight neutral polymers in order to calculate a fold-increase in antibody staining and analyze and compare data from different days. For guar gum, PVA, and PVP, a limited dilution series could be tested as higher concentrations of these solutions were too viscous to pipette accurately. As shown in FIGS. 2A to 2E, all of the high-molecular weight neutral polymers tested except PVP displayed an increase in antibody signal intensity on the array as a function of concentration and/or molecular weight relative to control incubations lacking these polymers. When compared to the fold-increase observed previously for 6.25% PEG- 20,000, however, only dextran-100 met or exceeded the results of the PEG-20,000.

### Example 4

### Analysis and Comparison of Antibody Diluent Buffers Comprising PEG-20,000 or Dextran-100

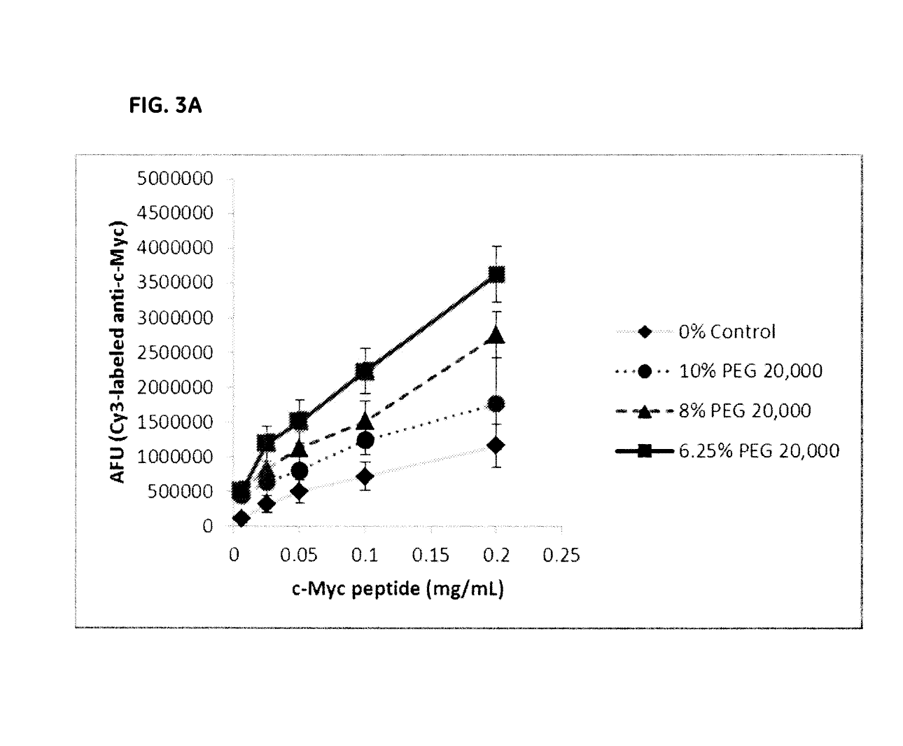

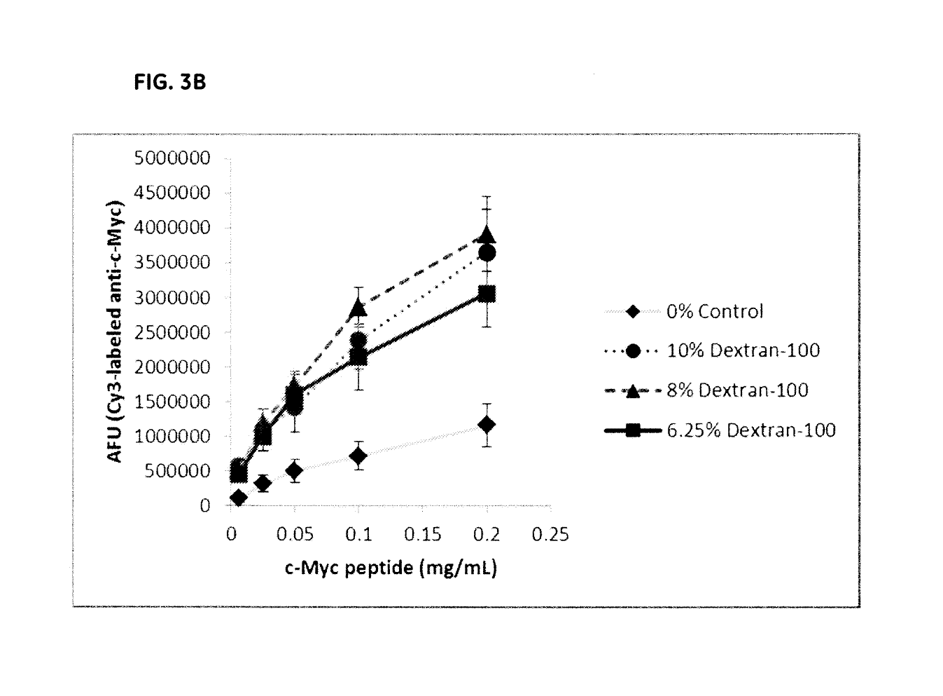

Based on evidence depicted in FIGS. 1 and 2E, wherein the optimal concentration ranges of dextran-100 or PEG-20,000 were determined to be around 6.25% (w/v) to 12.5% (w/v), antibody diluent buffer comprising either dextran-100 or PEG-20,000 were compared at intermediated concentration ranges (e.g., 8% (w/v) and 10% (w/v)) using the slide array immunostaining procedure outlined above. As shown in FIGS. 3A and 3B, these experiments clearly demonstrated that an antibody diluent buffer comprising dextran-100 has a broader performance range than that comprising PEG-20,000. Specifically, antibody diluent buffers comprising concentrations of PEG-20,000 greater than 6.25% (w/v) exhibited reductions in relative antibody intensity. In contrast, no loss in performance was observed with antibody diluent buffers comprising dextran-100 in excess of 6.25% (w/v), as depicted in FIG. 3B.

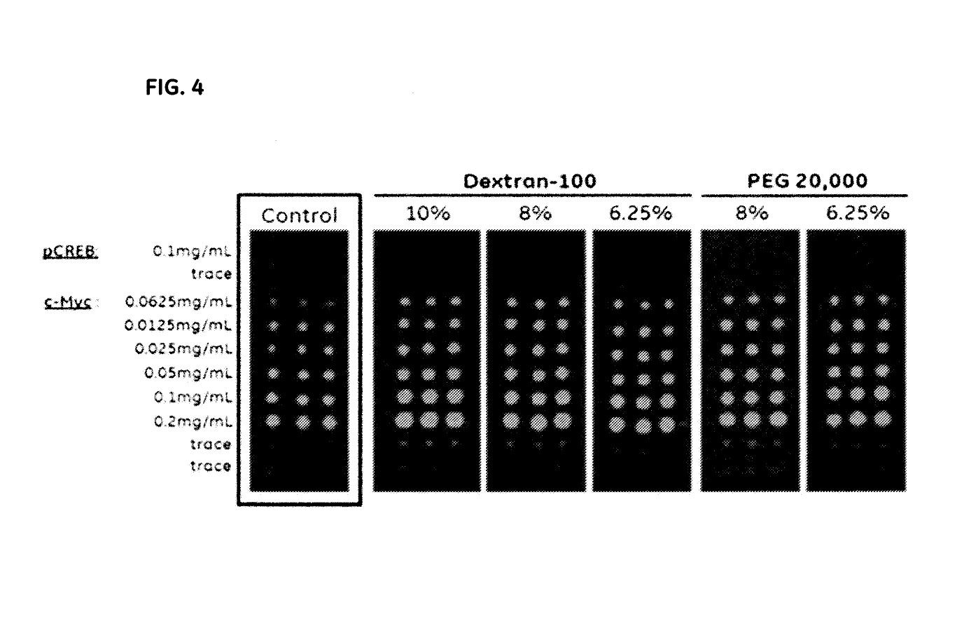

Representative Cy3 images of the microarrays immunostained in the absence or presence of dextran-100 or PEG 20,000 are shown in FIG. 4. Antibody diluent buffer comprising dextran-100 or PEG-20,000 permitted the detection of even trace amounts of c-Myc antigen in the SpotBot pin washes. This phenomenon was quantified in FIGS. 1-2E as well.

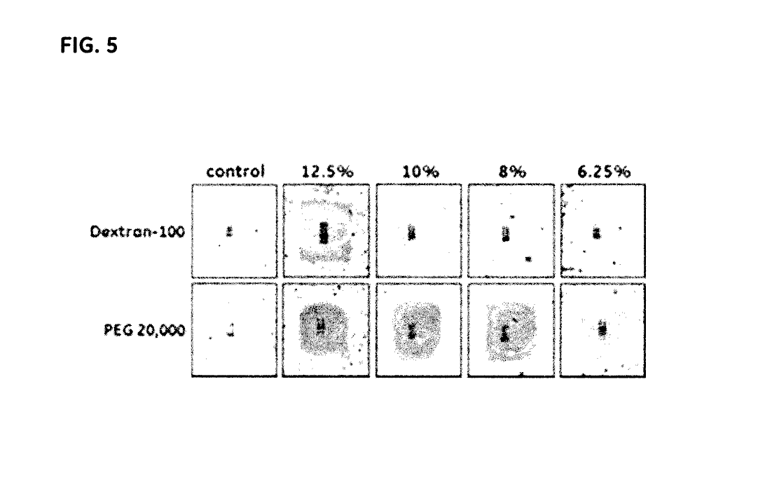

In addition to showing increased antibody signal intensity across a broader concentration range, the antibody diluent buffer comprising dextran-100 between 6.25% (w/v) and 10% (w/v) also exhibited less non-specific, background Cy3 fluorescence signal on the slide surface compared to antibody diluent buffers comprising PEG-20,000, as shown in FIG. 5. The undesirable increase in non-specific background fluorescence signal observed with antibody diluent buffers comprising PEG-20,000 may be a consequence of crowding-mediated antibody deposition (e.g., antibody aggregation) or inefficient removal of antibody during wash steps because of the elevated viscosity observed with antibody diluent buffers comprising PEG-20,000.

### Summary of Example Results

The results summarized in Examples 1-4 demonstrate the use of high-molecular weight neutral polymers as macromolecular crowding agents, particularly dextran-100, in antibody diluent buffers as a means to increase antibody signal intensity in a variety of antibody-based assays (e.g. immunoassays). The increase in immunostaining intensity conferred by the use of an antibody diluent buffer comprising dextran-100 ranged from approximately 2 to 20-fold depending on the antigen concentration analyzed. Antibody diluent buffers comprising dextran-100 permitted enhanced detection of even trace amounts of antigen. When applied as a passive crowding agent, dextran, particularly dextran-100, exhibited a much broader active concentration range (approximately 6-13% (w/v)) relative to PEG-20,000. Moreover, examples performed utilizing an antibody diluent comprising PEG-20,000 resulted in a higher nonspecific background fluorescence at PEG-20,000 concentrations above 6% (w/v) relative to analogous antibody diluent buffers comprising dextran-100. The results provided here strongly suggest that antibody diluents comprising high-molecular weight neutral polymers, particularly those having molecular weights of at least 25 kD, more particularly dextran (e.g., dextran-100), can be used in applications to reduce the amount of costly antibody preparations used in numerous in vitro and in situ antibody-based assays (e.g., immunoassays), including but not limited to immunostaining, immunolabeling, immunofluorescence, immunohistochemistry, flow cytometry, Western blot analysis, immunoelectron microscopy, a lateral flow immunoassay, and (ELISA). The cost savings from the use of antibody diluent buffers comprising high-molecular weight neutral polymers as macromolecular crowding agents could be sufficient to permit the performance of experiments that at present are cost-prohibitive.

### Example 5

### Antibody Staining of Tissue Samples

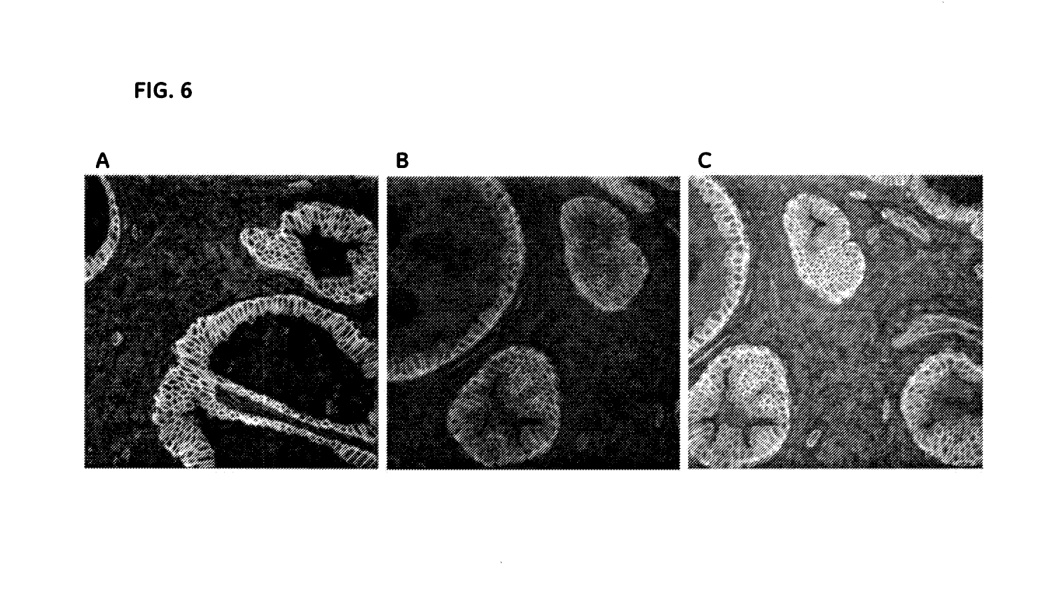

To extend this analysis beyond in vitro immunoassays, formalin-fixed paraffin embedded (FFPE) multi-tissue arrays were analyzed by standard clearing, hydration and antigen retrieval procedures, and then incubated in blocking solution (10% donkey serum, 3% BSA in PBS) overnight at 4° C. Slides were removed from the blocking solution, and 200 μL of antibody solution was applied to each slide (antibody in PBS+3% BSA) using the following antibodies: (1) 5 μg/mL polyclonal pan-cadherin antibody followed by Cy3-labeled donkey-anti-rabbit secondary or (2) 10 μg/mL polyclonal pan-cadherin antibody conjugated to Cy3. Alternatively, dextran-100 was diluted in PBS to 16% and mixed 1:1 with antibody staining solution to yield a final concentration of 10 μg/mL Cy3-conjugated pan-cadherin antibody and 8% dextran. The antibodies in the above-described antibody diluent buffers were incubated on the slides for 1 hour at room temperature in a humidified staining chamber shielded from direct light. Slides were carefully washed 3 times with PBS and imaged on an Olympus IX81 microscope. The results of these analyses are set forth in FIG. 6 and illustrate that antibody diluent buffer comprising dextran-100 noticeably improve antibody staining quality in FFPE tissue sections.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An antibody diluent buffer comprising a high-molecular weight neutral polymer having a molecular weight of at least 25 kilodaltons (kD) and a primary antibody, wherein the antibody is soluble and remains in solution.

2. An antibody diluent buffer comprising a high-molecular weight neutral polymer selected from the group consisting of a dextran, Ficoll, polyvinyl alcohol (PVA), guar gum, and hydroxyethyl starch (HES).

3. The antibody diluent buffer of claim 2, wherein the high-molecular weight neutral polymer is a dextran, and wherein the dextran has a molecular weight between 25 kD and 2000 kD.

4. The antibody diluent buffer of claim 3, wherein the dextran has a molecular weight between 40 kD and 100 kD.

5. The antibody diluent buffer of claim 3, wherein the dextran has a molecular weight is approximately 100 kD.

6. The antibody diluent buffer of claim 3, wherein the dextran is at a concentration of between 5% (w/v) and 15% (w/v).

7. The antibody diluent buffer of claim 6, wherein the dextran is at a concentration of between 6% (w/v) and 13% (w/v)

8. The antibody diluent buffer of claim 6, wherein the dextran is at a concentration of approximately 6.25% (w/v) or 12.5% (w/v).

9. The antibody diluent buffer of claim 1, wherein the primary antibody is labeled with a detectable substance.

10. The antibody diluent buffer of claim 9, wherein the detectable substance is selected from the group consisting of a fluorescent agent, an enzyme, a luminescent material, a bioluminescent material, a radioactive material, and nanoparticles.

11. A method for detecting an antigen in a sample comprising the steps of:
a) providing an antibody diluent buffer comprising a high-molecular weight neutral polymer and an antibody, wherein the antibody remains in solution;
b) incubating the antibody diluent buffer with a sample that may comprise the antigen;
c) determining if the antigen is present in the sample.

12. The method of claim 11, wherein the high-molecular weight neutral polymer is a dextran.

13. The method of claim 12, wherein the dextran has a molecular weight between 40 kD and 100 kD.

14. The method of claim 11, wherein the first antibody is labeled with a detectable substance and determining if the antigen is present in the sample comprises measuring for presence of the detectable substance.

15. The method of claim 11, wherein determining if the antigen is present in the sample comprises utilizing a secondary antibody that binds to a primary antibody, wherein the secondary antibody is labeled with a detectable substance and measuring for presence of the detectable substance to determine if the antigen is present in the sample.

16. The method of claim 11, wherein the method is an immunoassay.

17. The method of claim 16, wherein the immunoassay is selected from the group consisting of immunostaining, immunolabeling, immunofluorescence, immunohistochemistry, flow cytometry, Western blot analysis, immunoelectron microscopy, a lateral flow immunoassay, and an enzyme-linked immunosorbent assay (ELISA).

18. The method of claim 16, wherein use of the antibody diluent buffer improves the performance of the immunoassay.

19. The method of claim 11, wherein use of the antibody diluent buffer permits detection of a minimal amount of the antigen.

20. The method of claim 11, wherein use of the antibody diluent buffer reduces the concentration of the primary or a secondary antibody needed to detect the antigen in the sample.
